(19) United States
(12) Patent Application Publication
(10) Pub. No.: US 2013/0203861 A1
(43) Pub. Date: Aug. 8, 2013

(54) METHODS AND COMPOSITIONS FOR TREATING OVARIAN CANCER

(71) Applicant: Golden Biotechnology Corporation, New Taipei City (TW)

(72) Inventors: Sheng-Yung LIU, New Taipei City (TW); Wu-Che WEN, New Taipei City (TW); Mao-Tien KUO, New Taipei City (TW)

(73) Assignee: GOLDEN BIOTECHNOLOGY CORPORATION, New Taipei City (TW)

(21) Appl. No.: 13/828,237

(22) Filed: Mar. 14, 2013

Related U.S. Application Data
(63) Continuation-in-part of application No. 12/875,702, filed on Sep. 3, 2010.

(30) Foreign Application Priority Data
Sep. 9, 2009 (TW) 098130428

(51) Int. Cl. C07C 49/753

(52) U.S. Cl. CPC C07C 49/753
USPC 514/690; 435/375

(57) ABSTRACT

The present invention provides methods and compositions for treating ovarian cancer by cyclohexenone compounds.

### CROSS-REFERENCE

This application is a continuation-in-part application of Ser. No. 12/875,702, filed Sep. 3, 2010, which claims priority to Taiwan Application No. 098130428, filed Sep. 9, 2009, each of which is incorporated herein by reference in its entirety.

### BACKGROUND OF THE INVENTION

Ovarian cancer is the most common type of gynecological cancer. Reports indicate that the possible etiological factors of ovarian cancer include hormone and ovulation factors, environmental factors such as being exposed to carcinogens over a long period of time, family history, obesity, age and the existence of breast cancer, which creates a high risk for causing ovarian cancer, and so on. The symptoms of early stage ovarian cancer are lower abdominal discomfort, nausea and anorexia, which are similar to general gastroenteric disorders and not the obvious and identifiable symptoms, and cannot be detected by usual and simple diagnosis. Currently, there are no effective and precise diagnostic methods to detect early stage ovarian cancer. Thus, most cases could not be identified as ovarian cancer until the terminal stages of cancer. Approximately 70 percent of diagnosed ovarian cancer belongs to stages III-IV when the cancer cells often metastasize and spread out with extremely poor prognosis. As a result, the survival rate of female cancer patients is quite low, and ovarian cancer has already become one of the mainly death causing gynecological cancer in developed countries.

### SUMMARY OF THE INVENTION

In one aspect provides herein for treating or reducing the risk of ovarian cancer comprising administering to a subject a therapeutically effective amount of a cyclohexenone compound having the structure:

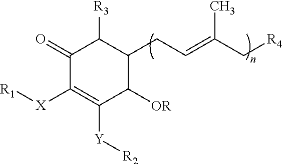

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In another aspect provides herein methods for inhibiting ovarian cancer cells comprising contacting the cancer cells a therapeutically effective amount of a compound having the structure:

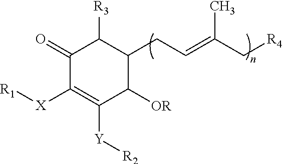

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The treatments of ovarian cancer include surgery, chemotherapy and radiation and/or combination thereof. The early stage ovarian cancer is treated by surgery plus preventive chemotherapy and the ovarian cancer in later stages must be treated by complete chemotherapy or radiotherapy to prevent the cancer cells to proliferate after surgery. Besides the active resection of excisable carcinogenic lesions in the case of recurrent ovarian cancer, the replacement of chemotherapy drugs can also increase the therapeutic efficacy. However, the treatment efficiency is not as good as expected due to the lack of early accurate diagnosis and thus the overall 5-year survival rate is only 30 percent. In addition, these treatments usually lead to adverse side effects or clinical uncomfortable symptoms. Therefore, the investigation and development of a therapeutic substance that can treat ovarian cancer with less or no side effects are in urgent demand.

In some embodiments, provided herein are methods for treating or reducing the risk of ovarian cancer by administering a cyclohexenone compound provided herein to a subject (e.g. a human). The cyclohexenone compounds provide therapeutic benefit to a subject being treated for ovarian cancer (see Examples 1-4). The invention cyclohexenone compounds, in some embodiments, are obtained from extracts of natural products and provide reduced complications and/or side effects. In some embodiments, this invention provides the therapeutic and prophylactic potential of exemplary cyclohexenone compounds (e.g., Compound 1) for treating or reducing the risk of ovarian cancer.

In some embodiments, there are provided methods for treating or reducing the risk of ovarian cancer comprising administering to a subject a therapeutically effective amount of a cyclohexenone compound having the structure:

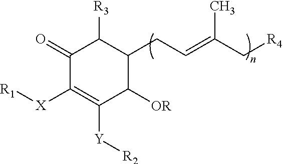

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In some embodiments, the compound reduces ovarian cancer tumor size or tumor volume. In certain embodiments, the compound decreases ovarian cancer tumor growth rate in a subject. In some embodiments, the compound inhibits the growth of ovarian cancer cells. In some embodiments, the subject is human. See Examples 2-4.

In some embodiments, the cyclohexenone compound having the structure

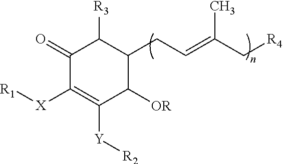

is prepared synthetically or semi-synthetically from any suitable starting material. In other embodiments, the cyclohexenone compound is prepared by fermentation, or the like. For example, Compound 1 (also known as Antroquinonol™ or “Antroq”) or Compound 3, in some instances, is prepared from 4-hydroxy-2,3-dimethoxy-6-methylcyclohexa-2,5-dienone. The non-limited exemplary compounds are illustrated below.

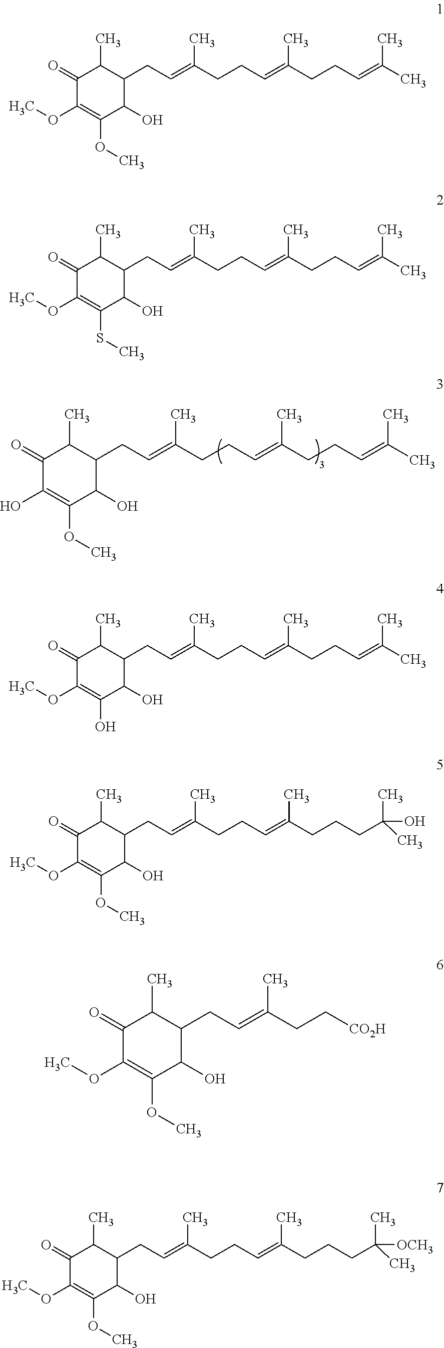

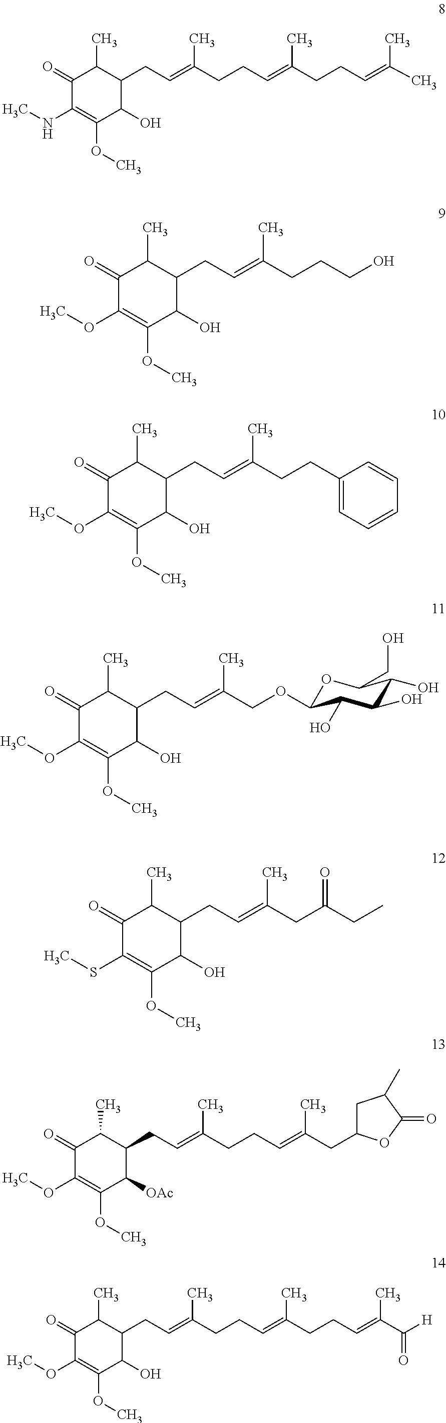

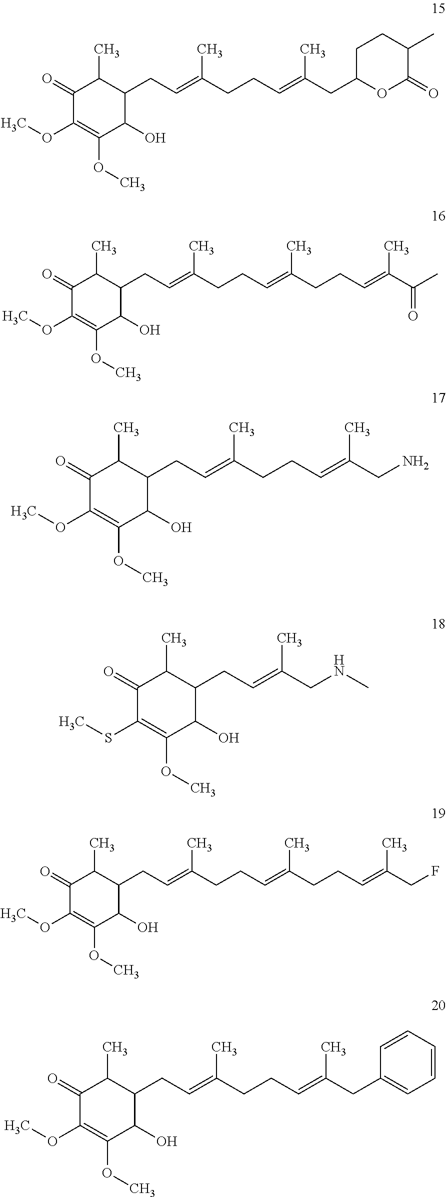

In other embodiments, the cyclohexenone compound having the structure

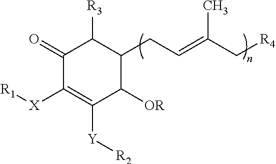

is isolated from the organic solvent extracts of Antrodia camphorata. In some embodiments, the organic solvent is selected from alcohols (e.g., methanol, ethanol, propanol, or the like), esters (e.g., methyl acetate, ethyl acetate, or the like), alkanes (e.g., pentane, hexane, heptane, or the like), halogenated alkanes (e.g., chloromethane, chloroethane, chloroform, methylene chloride, and the like), and the like. For example, exemplary Compounds 1-7 are isolated from organic solvent extracts. In certain embodiments, the organic solvent is alcohol. In certain embodiments, the alcohol is ethanol. In some embodiments, the cyclohexenone compound is isolated from the aqueous extracts of Antrodia camphorate.

In some embodiments, R is a hydrogen, C(═O)C₃H₈, C(═O)C₂H₅, or C(═O)CH₃. In some embodiments, R₁ is a hydrogen or methyl. In certain embodiments, R₂ is a hydrogen, methyl, ethyl, propyl, butyl, pentyl or hexyl. In some embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In some embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, C₂-C₈alkenyl, C₂-C₈alkynyl, aryl, and glucosyl, wherein 5 or 6-membered lactone, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂. In certain embodiments, the compound is

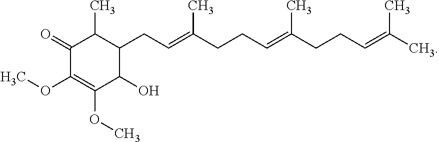 [SMILES: COC1=C(OC)C(O)C(C/C=C(\C)CC/C=C(\C)CCC=C(C)C)C(C)C1=O]

In some embodiments, there are provided methods for inhibiting ovarian cancer cells comprising contacting the cancer cells a therapeutically effective amount of a compound having the structure:

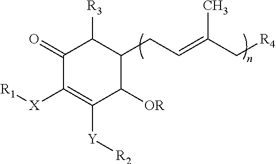

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In some embodiments, the ovarian cancer cells are derived from human cancer cells. In certain embodiments, the cancer cells are ES-2, or the like.

### Certain Pharmaceutical and Medical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl group may be a saturated alkyl group (which means that it does not contain any carbon-carbon double bonds or carbon-carbon triple bonds) or the alkyl group may be an unsaturated alkyl group (which means that it contains at least one carbon-carbon double bonds or carbon-carbon triple bond). The alkyl moiety, whether saturated or unsaturated, may be branched, or straight chain.

The “alkyl” group may have 1 to 12 carbon atoms (whenever it appears herein, a numerical range such as “1 to 12 refers to each integer in the given range; e.g., “1 to 12 carbon atoms” means that the alkyl group may consist of 1 carbon atom, 2 carbon atoms, 3 carbon atoms, etc., up to and including 12 carbon atoms, although the present definition also covers the occurrence of the term “alkyl” where no numerical range is designated). The alkyl group of the compounds described herein may be designated as “C₁-C₈ alkyl” or similar designations. By way of example only, “C₁-C₈ alkyl” indicates that there are one, two, three, four, five, six, seven or eight carbon atoms in the alkyl chain. In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, allyl, but-2-enyl, but-3-enyl, cyclopropylmethyl, cyclobutylmethyl, cyclopentylmethyl, cyclohexylmethyl, and the like. In one aspect, an alkyl is a C₁-C₈ alkyl.

The term “alkylene” refers to a divalent alkyl radical. Any of the above mentioned monovalent alkyl groups may be an alkylene by abstraction of a second hydrogen atom from the alkyl. In one aspect, an alkylene is a C₁-C₁₂alkylene. In another aspect, an alkylene is a C₁-C₈alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

As used herein, the term “aryl” refers to an aromatic ring wherein each of the atoms forming the ring is a carbon atom. Aryl rings are formed by five, six, seven, eight, nine, or more than nine carbon atoms. Aryl groups are optionally substituted. In one aspect, an aryl is a phenyl or a naphthalenyl. In one aspect, an aryl is a phenyl. In one aspect, an aryl is a C₆-C₁₀aryl. Depending on the structure, an aryl group can be a monoradical or a diradical (i.e., an arylene group). In one aspect, an arylene is a C₆-C₁₀ arylene. Exemplary arylenes include, but are not limited to, phenyl-1,2-ene, phenyl-1,3-ene, and phenyl-1,4-ene.

The term “aromatic” refers to a planar ring having a delocalized π-electron system containing 4n+2π electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “halo” or, alternatively, “halogen” or “halide” means fluoro, chloro, bromo or iodo.

The term “lactone” refers to a cyclic ester which can be seen as the condensation product of an alcohol group —OH and a carboxylic acid group —COOH in the same molecule. It is characterized by a closed ring consisting of two or more carbon atoms and a single oxygen atom, with a ketone group ═O in one of the carbons adjacent to the other oxygen.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 4 to 10 atoms in its ring system, and with the proviso that the any ring does not contain two adjacent O or S atoms. Non-aromatic heterocyclic groups (also known as heterocycloalkyls) include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles may be substituted with one or two oxo (═O) moieties, such as pyrrolidin-2-one.

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. In some embodiments, depending on the structure, an alkenyl group is a monoradical or a diradical (i.e., an alkenylene group). In some embodiments, alkenyl groups are optionally substituted. Illustrative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-cecenyl.

The term “alkynyl” as used herein, means a straight, branched chain, or cyclic (in which case, it would also be known as a “cycloalkenyl”) hydrocarbon containing from 2-10 carbons and containing at least one carbon-carbon triple bond formed by the removal of four hydrogens. In some embodiments, depending on the structure, an alkynyl group is a monoradical or a diradical (i.e., an alkynylene group). In some embodiments, alkynyl groups are optionally substituted. Illustrative examples of alkynyl include, but are not limited to, ethynyl, propynyl, butynyl, pentynyl, hexynyl, heptynyl, and the like.

The term “alkoxy” as used herein, means an alkyl group, as defined herein, appended to the parent molecular moiety through an oxygen atom. Illustrative examples of alkoxy include, but are not limited to, methoxy, ethoxy, propoxy, 2-propoxy, butoxy, tert-butoxy, pentyloxy, and hexyloxy.

The term “cycloalkyl” as used herein, means a monocyclic or polycyclic radical that contains only carbon and hydrogen, and includes those that are saturated, partially unsaturated, or fully unsaturated. Cycloalkyl groups include groups having from 3 to 10 ring atoms. Representative examples of cyclic include but are not limited to, the following moieties:

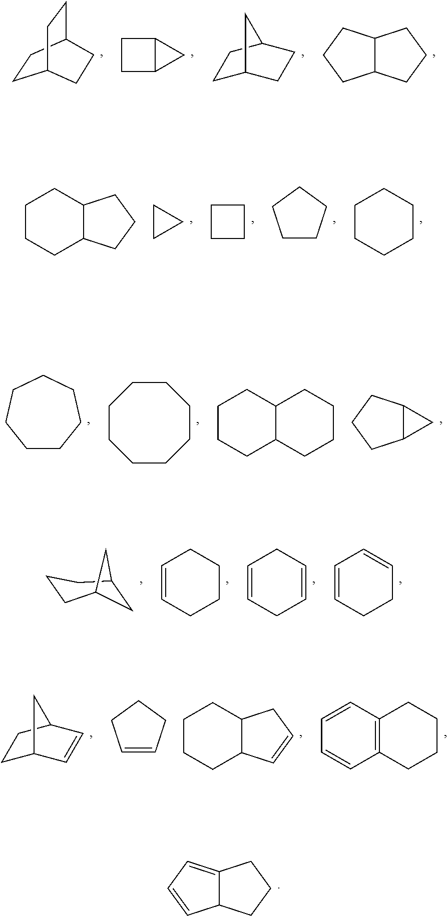

In some embodiments, depending on the structure, a cycloalkyl group is a monoradical or a diradical (e.g., a cycloalkylene group).

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “glucosyl” as used herein, include D- or L-form glucosyl groups, in which the glucosyl group is attached via any hydroxyl group on the glucose ring.

The term “acceptable” with respect to a formulation, composition or ingredient, as used herein, means having no persistent detrimental effect on the general health of the subject being treated.

Antrodia is a genus of fungi in the family Meripilaceae. Antrodia species have fruiting bodies that typically lie flat or spread out on the growing surface, with the hymenium exposed to the outside; the edges may be turned so as to form narrow brackets. Most species are found in temperate and boreal forests, and cause brown rot. Some of the species in this genus are have medicinal properties, and have been used in Taiwan as a Traditional medicine.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound (i.e., a cyclohexenone compound described herein) and a co-agent, are both administered to a patient simultaneously in the form of a single entity or dosage. The term “non-fixed combination” means that the active ingredients, e.g. a compound (i.e., a cyclohexenone compound described herein) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound (i.e., a cyclohexenone compound described herein) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments, the cyclohexenone compound, or a pharmaceutically acceptable salt, metabolite, solvate or prodrug thereof, is administered parenterally or intravenously. In other embodiments, the cyclohexenone compound, or a pharmaceutically acceptable salt, metabolite, solvate or prodrug thereof, is administered by injection. In some embodiments, the cyclohexenone compound, or a pharmaceutically acceptable salt, metabolite, solvate or prodrug thereof, is administered orally.

### Pharmaceutical Formulation

In some embodiments provide pharmaceutical compositions comprising a therapeutically effective amount of a cyclohexenone compound having the structure:

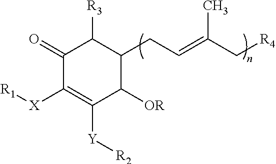

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof; and a pharmaceutically acceptable excipient.

In some embodiments, the cyclohexenone compounds of the pharmaceutical compositions have the structure:

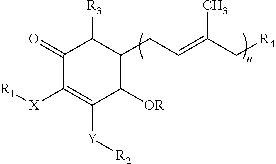

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof.

In some embodiments, R is a hydrogen, C(═O)C₃H₈, C(═O)C₂H₅, or C(═O)CH₃. In some embodiments, each of R₁, R₂ and R₃ independently is a hydrogen, methyl, ethyl, propyl, butyl, pentyl hexyl, heptyl, or octyl. In certain embodiments, R₁ is a hydrogen or methyl. In certain embodiments, R₂ is a hydrogen, methyl, ethyl, propyl, butyl, pentyl or hexyl. In certain embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, C₁-C₈alkyl, aryl, or glucosyl, wherein the 5 or 6-membered lactone, C₁-C₈alkyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, C₁-C₈alkyl, aryl, or glucosyl, wherein the 5 or 6-membered lactone, C₁-C₈alkyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

In certain embodiments, the compound is selected from group consisting of

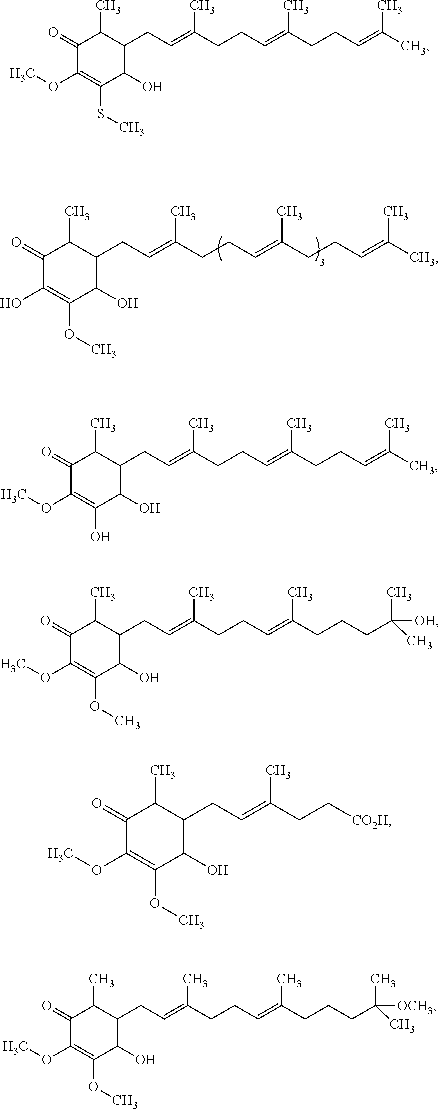

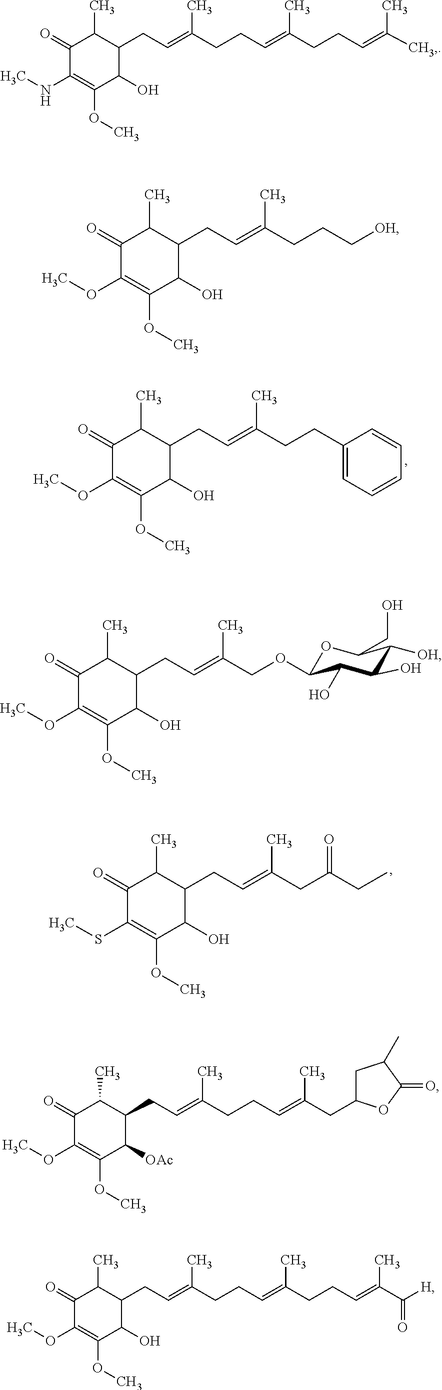

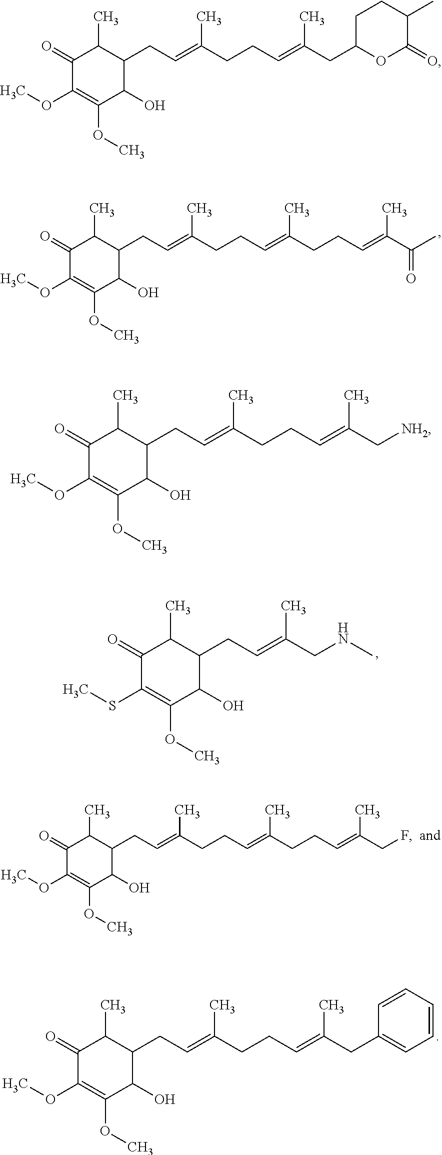

In certain embodiments, the compound is selected from group consisting of

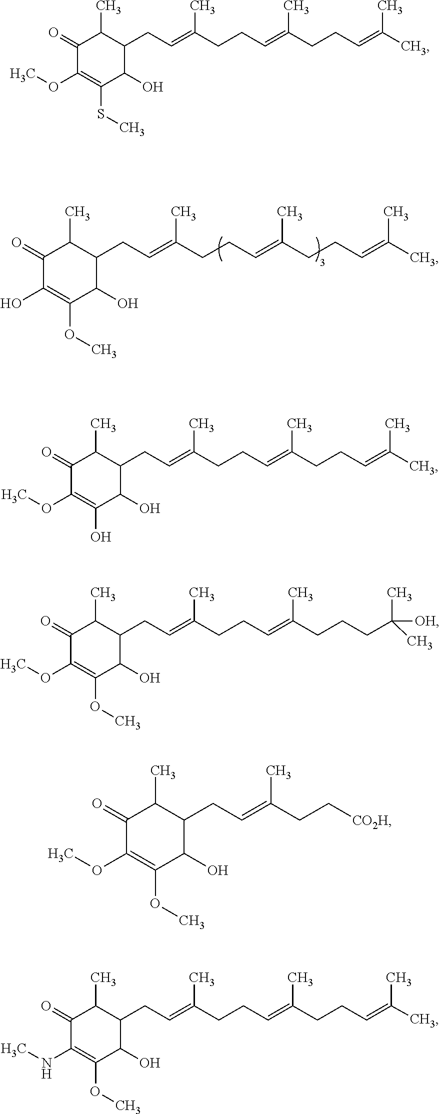

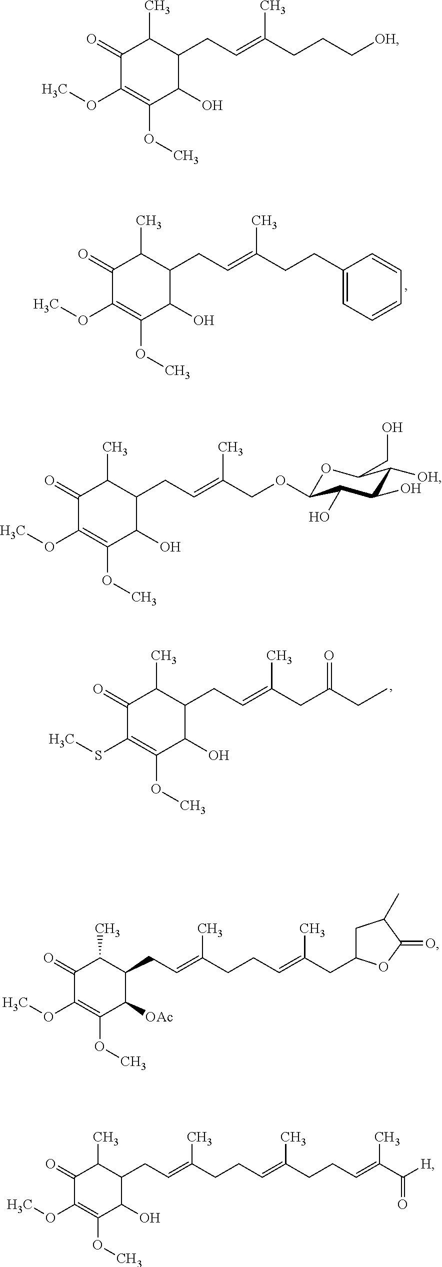

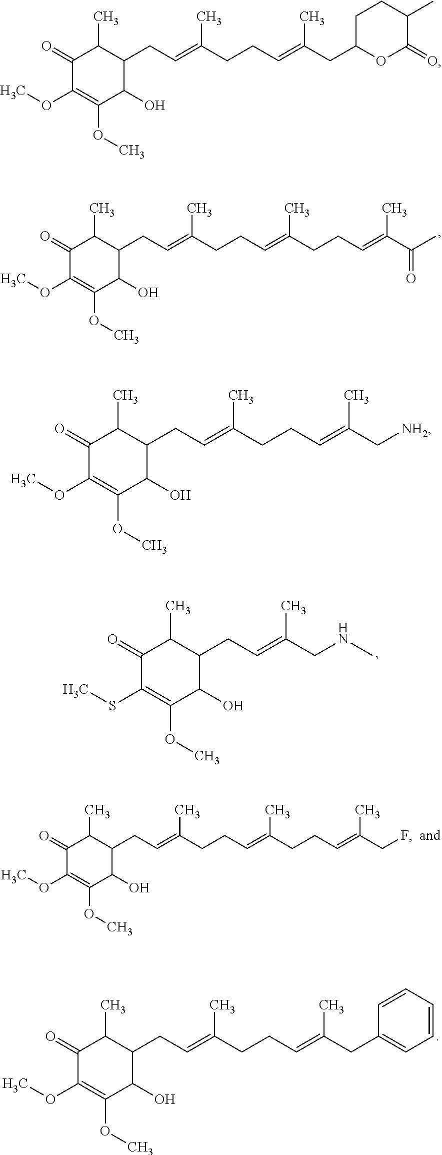

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound (i.e., a cyclohexenone compound described herein) and a pharmaceutically acceptable diluent(s), excipient(s), or carrier(s). In certain embodiments, the compounds described are administered as pharmaceutical compositions in which a compound (i.e., a cyclohexenone compound described herein) is mixed with other active ingredients, as in combination therapy. Encompassed herein are all combinations of actives set forth in the combination therapies section below and throughout this disclosure. In specific embodiments, the pharmaceutical compositions include one or more compounds (i.e., a cyclohexenone compound described herein).

A pharmaceutical composition, as used herein, refers to a mixture of a compound (i.e., a cyclohexenone compound described herein) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds (i.e., a cyclohexenone compound described herein) are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, a compound (i.e., a cyclohexenone compound described herein) is formulated in an aqueous solution. In specific embodiments, the aqueous solution is selected from, by way of example only, a physiologically compatible buffer, such as Hank's solution, Ringer's solution, or physiological saline buffer. In other embodiments, a compound (i.e., a cyclohexenone compound described herein) is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including a compound (i.e., a cyclohexenone compound described herein), are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions of a compound (i.e., a cyclohexenone compound described herein) are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, compounds (i.e., cyclohexenone compounds described herein) are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes a compound (i.e., a cyclohexenone compound described herein) to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL, 0.6 mL, 1.0 mL or other suitable volume of solution at about a concentration of 0.5 mg to 50 mg of a compound (i.e., a cyclohexenone compound described herein) per 1 mL of solution. Each injector is capable of delivering only one dose of the compound.

In still other embodiments, the compounds (i.e., cyclohexenone compounds described herein) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds (i.e., cyclohexenone compounds described herein) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of a compound (i.e., a cyclohexenone compound described herein) is accomplished by means of iontophoretic patches and the like. In certain embodiments, transdermal patches provide controlled delivery of a compound (i.e., a cyclohexenone compound described herein). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of a compound (i.e., a cyclohexenone compound described herein); (2) a penetration enhancer; and (3) an aqueous adjuvant. In addition, transdermal formulations can include additional components such as, but not limited to, gelling agents, creams and ointment bases, and the like. In some embodiments, the transdermal formulations further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compounds (i.e., cyclohexenone compounds described herein) are formulated for administration by inhalation. Various forms suitable for administration by inhalation include, but are not limited to, aerosols, mists or powders. Pharmaceutical compositions of a compound (i.e., a cyclohexenone compound described herein) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatins for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated herein by reference. Formulations, which include a compound (i.e., a cyclohexenone compound described herein), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

In still other embodiments, the compounds (i.e., cyclohexenone compounds described herein) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising a compound (i.e., a cyclohexenone compound described herein) may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound (i.e., cyclohexenone compounds described herein) described herein as an active ingredient. The active ingredient is in free-acid or free-base form, or in a pharmaceutically acceptable salt form. In addition, the methods and pharmaceutical compositions described herein include the use crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least compound (i.e., cyclohexenone compounds described herein) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of a compound (i.e., cyclohexenone compounds described herein). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### Combination Treatments

In general, the compositions described herein and, in embodiments where combinational therapy is employed, other agents do not have to be administered in the same pharmaceutical composition, and in some embodiments, because of different physical and chemical characteristics, are administered by different routes. In some embodiments, the initial administration is made according to established protocols, and then, based upon the observed effects, the dosage, modes of administration and times of administration is modified by the skilled clinician.

In some embodiments, therapeutically-effective dosages vary when the drugs are used in treatment combinations. Combination treatment further includes periodic treatments that start and stop at various times to assist with the clinical management of the patient. For combination therapies described herein, dosages of the co-administered compounds vary depending on the type of co-drug employed, on the specific drug employed, on the disease, disorder, or condition being treated and so forth.

It is understood that in some embodiments, the dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in other embodiments, the dosage regimen actually employed varies widely and therefore deviates from the dosage regimens set forth herein. Combinations of compounds (i.e., the cyclohexenone compound described herein) with other anti-cancer therapeutic agents, especially anti-ovarian cancer, are intended to be covered. In some embodiments, examples of anti-ovarian cancer agents include, but are not limited to, the following: carboplatin (Paraplatin), cisplatin, taxane s such as paclitaxel (Taxol) or docetaxel (Taxotere), and the like.

The combinations of the cyclohexenone compounds and other ovarian cancer therapeutic agents described herein encompass additional therapies and treatment regimens with other agents in some embodiments. Such additional therapies and treatment regimens can include another ovarian cancer therapy in some embodiments. Alternatively, in other embodiments, additional therapies and treatment regimens include other agents used to treat adjunct conditions associated with the ovarian cancer or a side effect from such agent in the combination therapy. In further embodiments, adjuvants or enhancers are administered with a combination therapy described herein.

In some embodiments provide compositions for the treatment of ovarian cancer comprising a therapeutically effective amount of a cyclohexenone compound having the structure:

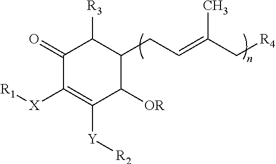

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof; and one or more ovarian cancer therapeutic agents.

### EXAMPLES

### Example 1

### Preparation of the Exemplary Cyclohexenone Compounds

One hundred grams of mycelia, fruiting bodies or mixture of both from Antrodia camphorata were placed into a flask. A proper amount of water and alcohol (70-100% alcohol solution) was added into the flask and were stirred at 20-25° C. for at least 1 hour. The solution was filtered through a filter and 0.45 μm membrane and the filtrate was collected as the extract.

The filtrate of Antrodia camphorata was subjected to High Performance Liquid chromatography (HPLC) analysis. The separation was performed on a RP18 column, the mobile phase consisted of methanol (A) and 0.3% acetic acid (B), with the gradient conditions of 0-10 min in 95%-20% B, 10-20 min in 20%-10% B, 20-35 min in 10%-10% B, 35-40 min in 10%-95% B, at the flow rate of 1 ml/min. The column effluent was monitored with a UV-visible detector.

The fractions collected at 21.2 to 21.4 min were collected and concentrated to yield compound 5, a product of pale yellow liquid. Compound 5 was analyzed to be 4-hydroxy-5-(11-hydroxy-3,7,11-trimethyldodeca-2,6-dienyl)-2,3-dimethoxy-6-methylcyclohex-2-enone with molecular weight of 408 (Molecular formula: C₂₄H₄₀O₅). ¹H-NMR (CDCl₃) δ (ppm)=1.21, 1.36, 1.67, 1.71, 1.75, 1.94, 2.03, 2.07, 2.22, 2.25, 3.68, 4.05, 5.71 and 5.56. ¹³C-NMR (CDCl₃) δ (ppm): 12.31, 16.1, 16.12, 17.67, 25.67, 26.44, 26.74, 27.00, 30.10, 40.27, 43.34, 59.22, 60.59, 71.8, 120.97, 123.84, 124.30, 131.32, 134.61, 135.92, 138.05, 160.45, and 197.11.

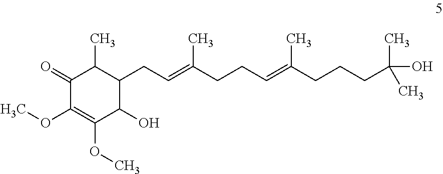 [SMILES: COC1=C(OC)C(O)C(C/C=C(\C)CC/C=C(\C)CCCC(C)(C)O)C(C)C1=O]

### Compound 5: 4-hydroxy-5-(11-hydroxy-3,7,11-trimethyldodeca-2,6-dienyl)-2,3-dimethoxy-6-methylcyclohex-2-enone

The fractions collected at 23.7 to 24.0 min were collected and concentrated to yield compound 7, a product of pale yellow liquid. Compound 7 was analyzed to be 4-hydroxy-2,3-dimethoxy-5-(11-methoxy-3,7,11-trimethyldodeca-2,6-dienyl)-6-methylcyclohex-2-enone with molecular weight of 422 (C₂₅H₄₂O₅). ¹H-NMR (CDCl₃) δ (ppm)=1.21, 1.36, 1.71, 1.75, 1.94, 2.03, 2.07, 2.22, 2.25, 3.24, 3.68, 4.05, 5.12, 5.50, and 5.61. ¹³C-NMR (CDCl₃) δ (ppm): 12.31, 16.1, 16.12, 17.67, 24.44, 26.44, 26.74, 27.00, 37.81, 39.81, 40.27, 43.34, 49.00, 59.22, 60.59, 120.97, 123.84, 124.30, 135.92, 138.05, 160.45 and 197.12.

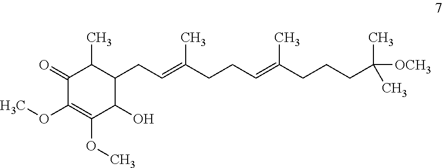 [SMILES: COC1=C(OC)C(O)C(C/C=C(\C)CC/C=C(\C)CCCC(C)(C)OC)C(C)C1=O]

### Compound 7: 4-hydroxy-2,3-dimethoxy-5-(11-methoxy-3,7,11-trimethyldodeca-2,6-dienyl)-6-methylcyclohex-2-enone

The fractions collected at 25 to 30 min were collected and concentrated to yield 4-hydroxy-2,3-dimethoxy-6-methyl-5-(3,7,11-trimethyldodeca-2,6,10-trienyl)cyclohex-2-enone (compound 1), a product of pale yellow brown liquid. The analysis of compound 1 showed the molecular formula of C₂₄H₃₈O₄, molecular weight of 390 with melting point of 48 to 52° C. NMR spectra showed that ¹H-NMR (CDCl₃) δ (ppm)=1.51, 1.67, 1.71, 1.75, 1.94, 2.03, 2.07, 2.22, 2.25, 3.68, 4.05, 5.07, and 5.14; ¹³C-NMR (CDCl₃) δ (ppm)=12.31, 16.1, 16.12, 17.67, 25.67, 26.44, 26.74, 27.00, 39.71, 39.81, 40.27, 43.34, 59.22, 60.59, 120.97, 123.84, 124.30, 131.32, 135.35, 135.92, 138.05, 160.45, and 197.12.

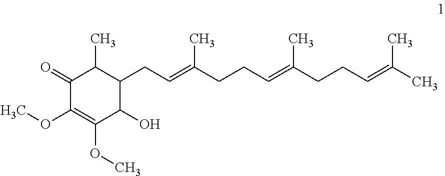 [SMILES: COC1=C(OC)C(O)C(C/C=C(\C)CC/C=C(\C)CCC=C(C)C)C(C)C1=O]

### Compound 1: 4-hydroxy-2,3-dimethoxy-6-methyl-5-(3,7,11-trimethyldodeca-2,6,10-trienyl)cyclohex-2-enone

Compound 6, a metabolite of compound 1, was obtained from urine samples of rats fed with Compound 1 in the animal study. Compound 6 was determined to be 4-hydroxy-2,3-dimethoxy-6-methyl-5-(3-methyl-2-hexenoic acid)cyclohex-2-enone with molecular weight of 312 (C₁₆H₂₄O₆). Compound 4 which was determined as 3,4-dihydroxy-2-methoxy-6-methyl-5-(3,7,11-trimethyldodeca-2,6,10-trienyl)cyclohex-2-enone (molecular weight of 376, C₂₃H₃₆O₄), was obtained when compound 1 was under the condition of above 40° C. for 6 hours.

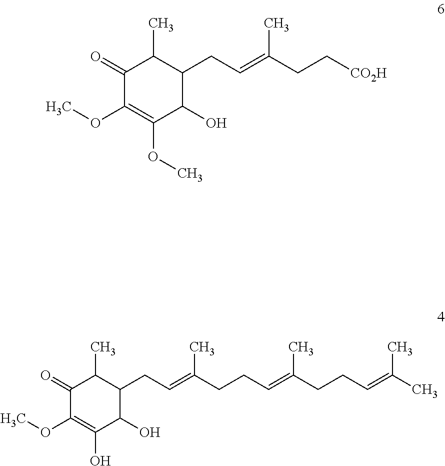 [SMILES: COC1=C(OC)C(O)C(C/C=C(\C)CCC(=O)O)C(C)C1=O ; COC1=C(O)C(O)C(C/C=C(\C)CC/C=C(\C)CCC=C(C)C)C(C)C1=O]

Alternatively, the exemplary compounds may be prepared from 4-hydroxy-2,3-dimethoxy-6-methylcyclohexa-2,5-dienone, or the like.

Similarly, other cyclohexenone compounds having the structure

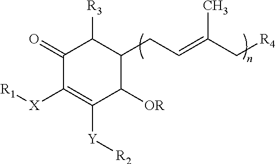

are isolated from Antrodia camphorata or prepared synthetically or semi-synthetically from the suitable starting materials. An ordinary skilled in the art would readily utilize appropriate conditions for such synthesis.

### Example 2

### MTT Assay for Anti-Ovarian Cancer

Exemplary Compound 1 was added into the culture media of ovarian cancer cell line ES-2 (a human ovarian cancer cell line) to determine the survival rates. Survival of cells was analyzed using MTT assay. Other suitable human ovarian cancer cell lines may be used by a skilled person in the art.

MTT assay is commonly used to analyze cell proliferation, survival rate of viable cells and cytotoxicity. MTT (3[4,5-dimethylthiazol-2-yl]-2,5-diphenyltetrazolium bromide) is a yellow dye which can be converted to water-insoluble purple formazan on the reductive cleavage of its tetrazolium ring by the succinate tetrazolium reductase in mitochondria of cells. The amount of formazan produced is used to detect the number of viable cells and calculate the survival rates.

The ES-2 cells were cultivated in McCoy' 5 A medium supplemented with 10% fetal bovine serum, 10 U/ml of Penicillin and 100 μg/ml of Streptomycin at 37° C., 5% CO₂ for 24 hours. Proliferated cells were washed once with PBS, treated with 1× trypsin-EDTA, and centrifuged at 1200 rpm for 5 min. The supernatant was removed and the cell pellet was resuspended in 10 ml of fresh medium by gently shaking. Cells were seeded onto 96-well plates. Cells treated with the crude extracts of Antrodia camphorata (total ethanol extracts, not purified) were designed as the control group; and cells treated with Compound 1 were designed as the experiment group. Both substrates were added in the concentration of 30, 10, 3, 1, 0.3, 0.1 and 0.03 μg/ml respectively. Cells were cultivated at 37° C., 5% CO₂ for 48 hours. Afterward, 2.5 mg/ml of MTT solution was added to each well and incubated in the dark for 4 hours, followed by the addition of 100 μl of lysis buffer to stop the reaction. The absorbances were measured at 570 nm with an ELISA Reader to determine the survival rates. The half inhibition concentration (IC₅₀) value was also calculated and listed in Table 1.

TABLE 1
Results of MTT assay for inhibition of ovarian cancer cells by Compound 1

| Sample | IC₅₀ (μg/ml) |
|---|---|
| Experiment group (Compound 1) | 0.80 |
| ES-2 | |

Refers to the result of table 1, the IC₅₀ value of Compound 1 toward ES-2 was 0.80 μg/ml, which was significantly lower than those of total extracts from Antrodia camphorate (data not shown). It is clearly shown that the cyclohexenone compounds (e.g., the exemplary Compound 1) provided herein inhibit ovarian cancer cells.

### Example 3

### Animal Model Study of Compound 1 in Treating Ovarian Cancer

### Animals

All animal experiments are done according to an Institutional Animal Care and Use Committee-approved protocol. Female nude mice are used between the ages of 6 and 7 weeks. Before injecting the ovarian cancer cells (e.g., ES-2 or A2780) into animals, the cells are washed twice, counted, and resuspended in PBS at 2×10⁶/100 μl. A2780 cells (2×10⁶ suspended in 100 μl of PBS) are inoculated into the intraperitoneal cavity of mice (day 0). Treatment with Compound 1 is started 7 days after inoculation of the cells. Compound 1 is dissolved in corn oil to attain the dosages of 100 and 200 mg/kg body weight. The oil is changed daily and measured for oil intake. Compound 1 treatment is continued for the next 3 weeks until the mice are killed at 4 weeks. The mice are monitored daily for any discomfort and weighed every third day to check for tumor growth.

### Mitotic Count

Blood is collected in heparin-coated tubes just before mice are killed. Plasma isolated from blood of 6 mice from each group is subjected to analysis of a panel of hepatic function tests (aspartate aminotransferase [AST], alanine aminotransferase [ALT], albumin), kidney function tests (creatinine, urea, albumin), and glucose. All assays are performed using kits from Bioassay Systems (Hayward, Calif.). All assays are performed according to the manufacturer's instructions.

### Live Tumor Measurements

The maximum diameter of viable tumor is calculated by summing the largest unidimensional diameter of each fragment of tumor using the Olympus BX-41 microscope and a micrometer. Similarly, necrotic areas are measured, and the composite live tumor size is calculated from each slide.

### Example 4

### Open Trial of Compound 1 in Women with Advanced Ovarian Cancer

This study is designed to find out how effective and safe Compound 1, is in the treatment of epithelial ovarian cancer.

The study design is a non-randomized, open label, single center trial. Eligible patients are women who have a confirmed diagnosis of ovary, fallopian tube cancer or primary peritoneal serous papillary carcinoma who have relapsed or are refractory to therapy after primary treatment of their disease.

Study Type: Interventional

### Study Design: Allocation: Non-Randomized

Endpoint Classification: Safety/Efficacy Study

Intervention Model: Single Group Assignment

Masking: Open Label

Primary Purpose Treatment

### Primary Outcome Measures:

Progression Free Survival in Patients With Platinum and Taxane Refractory Ovarian Cancer, Fallopian Tube Cancer and Primary Peritoneal Cancer With Compound 1 Treatment. [Time Frame: life of the study] [Designated as safety issue: No]

Progression is defined using Response Evaluation Criteria In Solid Tumors Criteria or Cancer Antigen (CA)125 response using the modified Gynecologic Cancer Intergroup (GCIG) criteria.

Overall Survival in Patients With Platinum and Taxane Refractory Ovarian Cancer, Fallopian Tube Cancer and Primary Peritoneal Cancer With Compound 1 Treatment. [Time Frame: Life of study] [Designated as safety issue: No]

### Secondary Outcome Measures:

Toxicities of Patients Treated With Compound 1. [Time Frame: Life of the study] [Designated as safety issue: Yes]

Grade 4 Toxicity

| Arms | Assigned Interventions |
|---|---|
| Experimental: Compound 1 | Drug: Compound 1 |
| | Dosage form: 50 mg and 100 mg capsules |
| | Dosage levels: 50 mg, 100 mg, 200 mg, |
| | 300 mg, 450 mg, 600 mg, 800 mg and |
| | 900 mg (8 cohorts) |
| | Frequency: take daily for 4 weeks per |
| | subject per dosage level |
| Placebo Comparator: | Drug: Placebo |
| Placebo | Take daily for 4 weeks per subject per |
| | dosage level |

### Eligibility

Ages Eligible for Study: 18 Years and older

Genders Eligible for Study: Female

### Criteria

### Inclusive Criteria

1. Patients must have histologically or pathologically confirmed diagnosis of epithelial carcinoma of the ovary, fallopian tube cancer or primary peritoneal serous papillary carcinoma. Borderline ovarian tumors are not allowed.

2. Patients must have relapsed within 6 months of completing, or had a best response of increasing disease during any number of prior chemotherapy regimens with a platinum (either cisplatin or carboplatin) and a taxane (paclitaxel or docetaxel). These agents may have been administered concurrently or sequentially. Any number of additional regimens for recurrent disease will be allowed, as long as the patient performance status is 0-2 Gynecologic Oncology Group (GOG).

3. Patients must have measurable or evaluable (i.e. positive serum Cancer Antigen (CA)-125 marker) disease. Scans or physical examinations used for tumor measurement must have been completed within 28 days prior to registration. Scans or ultrasounds for non-measurable disease must have been performed within 28 days prior to registration.

4. Prior radiation is allowed as long as it encompassed no more than 25% of the bone marrow. Debulking surgery for relapsed disease is allowed as long as the patient has measurable or evaluable disease remaining after the surgery. Patient must have recovered from all side effects of surgery.

5. Patients must not have received chemotherapy, biologic therapy or any other investigational drug for any reason within 28 days prior to registration. Patients must not have had a major surgery within 14 days prior to registration.

6. Patients must have a GOG performance status of 0-2.

7. Patients must have adequate liver function as defined by a serum bilirubin≦2.0× the institutional upper limit of normal (IULN), serum glutamic-oxaloacetic transaminase (SGOT) or serum glutamic-pyruvic transaminase (SGPT)≦2.5× the institutional upper limit of normal obtained within 14 days prior to registration.

8. Patients must have an adequate renal function as defined by a serum creatinine≦1.5× the institutional upper limit of normal obtained within 14 days prior to registration

9. Patients must not have Class 3/4 cardiac problems as defined by the New York Heart Association Criteria (e.g., congestive heart failure, myocardial infarction within 2 months of study)

10. No other prior malignancy is allowed except for the following: adequately treated basal cell or squamous cell skin cancer, in situ cervical cancer, adequately treated Stage I or II cancer from which the patient is currently in complete remission, or any other cancer from which the patient has been disease-free for 5 years.

11. Patients must have the following hematological criteria: Hemoglobin of ≧9 gm/dL White blood cell count≧2500 Platelets≧100,000

12. Patients must be ≧18 years of age.

### Exclusion Criteria:

No borderline ovarian tumors and mixed mesodermal soft tissue sarcomas

No psychological, familial, sociological, or geographical conditions that do not permit medical follow-up or compliance with the study protocol

Except for cancer-related abnormalities, patients should not have unstable or preexisting major medical conditions

No medical life-threatening complications of their malignancies

No known severe and/or uncontrolled concurrent medical disease (e.g., uncontrolled diabetes, uncontrolled chronic renal or liver disease, active uncontrolled infection, or HIV)

Inadequately controlled hypertension (defined as systolic blood pressure≧150 and/or diastolic blood pressure≧100 mmHg on antihypertensive medications)

New York Heart Association (NYHA) Grade III or greater congestive heart failure

Evidence of 5 to ≦10% loss of weight from baseline (baseline defined as the screening weight taken approximately 14 days of Day 0) that is not related to ascites or paracentesis.

Evidence of uncontrollable nausea

Presence of central nervous system or brain metastases

### Example 5

### Oral Formulation

To prepare a pharmaceutical composition for oral delivery, 100 mg of an exemplary Compound 1 was mixed with 100 mg of corn oil. The mixture was incorporated into an oral dosage unit in a capsule, which is suitable for oral administration.

In some instances, 100 mg of a compound described herein is mixed with 750 mg of starch. The mixture is incorporated into an oral dosage unit for, such as a hard gelatin capsule, which is suitable for oral administration.

### Example 6

### Sublingual (Hard Lozenge) Formulation

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 7

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating or reducing the risk of ovarian cancer comprising administering to a subject a therapeutically effective amount of a compound having the structure: 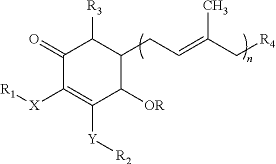
wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl,
wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

2. The method of claim 1, wherein the compound reduces ovarian cancer tumor size or tumor volume.

3. The method of claim 1, wherein the compound decreases ovarian cancer tumor growth rate.

4. The method of claim 1, wherein said compound inhibits the growth of ovarian cancer cells.

5. The method of claim 1, wherein said compound, or a pharmaceutically acceptable salt, metabolite, solvate or prodrug thereof, is administered orally, parenterally or intravenously.

6. The method of claim 1, wherein said compound, or a pharmaceutically acceptable salt, metabolite, solvate or prodrug thereof, is administered by injection.

7. The method of claim 1, wherein said compound, or a pharmaceutically acceptable salt, metabolite, solvate or prodrug thereof, is administered orally.

8. The method of claim 1, wherein said subject is human.

9. The method of claim 1, wherein said compound is isolated from Antrodia camphorate.

10. The method of claim 1, wherein R is a hydrogen, C(═O)C₃H₈, C(═O)C₂H₅, or C(═O)CH₃.

11. The method of claim 1, wherein each of R₁, R₂ and R₃ independently is a hydrogen, methyl, ethyl, propyl, butyl, pentyl, hexyl, heptyl, or octyl.

12. The method of any one of claim 11, wherein R₁ is a hydrogen or methyl.

13. The method of any one of claim 12, wherein R₂ is a hydrogen or methyl.

14. The method of claim 1, wherein R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, C(═O)NHC₂H₅, or OC(═O)NH₂.

15. The method of claim 1, wherein R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, C₁-C₈alkyl, aryl, or glucosyl, wherein the 5 or 6-membered lactone, C₁-C₈alkyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

16. The method of claim 15, wherein R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

17. The method of claim 16, wherein R₄ is CH₂CH═C(CH₃)₂.

18. The method of claim 1, wherein said compound is

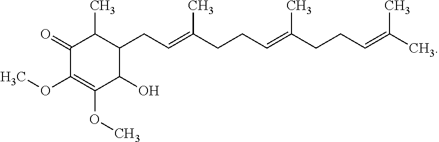 [SMILES: COC1=C(OC)C(O)C(C/C=C(\C)CC/C=C(\C)CCC=C(C)C)C(C)C1=O]

19. A method for inhibiting ovarian cancer cells comprising contacting the cancer cells a therapeutically effective amount of a compound having the structure: 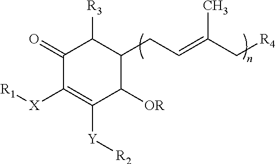
wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

20. The method of claim 19, wherein said compound is 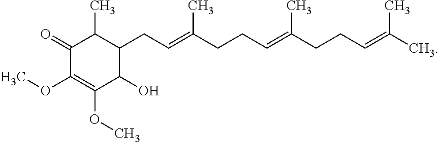 [SMILES: COC1=C(OC)C(O)C(C/C=C(\C)CC/C=C(\C)CCC=C(C)C)C(C)C1=O]
